(19) United States
(12) Patent Application Publication
(10) Pub. No.: US 2003/0113913 A1
(43) Pub. Date: Jun. 19, 2003

(54) Method of enhancing self renewal of stem cells and uses thereof

(75) Inventors: Louise Elizabeth Purton, Ascot Vale (AU); David Norman Haylock, Melbourne (AU); Paul John Simmons, Kew (AU); Steven James Collins, Seattle, WA (US)

Correspondence Address: SUGHRUE MION, PLLC, 2100 Pennsylvania Avenue, NW, Washington, DC, 20037-3213, US

(73) Assignee: PETER MACCALLUM CANCER INSTITUTE

(21) Appl. No.: 10/274,054

(22) Filed: Oct. 21, 2002

(30) Foreign Application Priority Data
Nov. 16, 2001 (AU) PR8925

(51) Int. Cl. C12N 5/08

(52) U.S. Cl. 435/372

(57) ABSTRACT

The present invention relates to methods for self renewal of stem cells. In particular the invention relates to stem cells of increased transplant potential or enhanced self renewal, stem cell cultures derived therefrom and uses of the stem cell cultures for treatment and particularly for transplantation and gene therapy protocols.

In a first aspect of the present invention there is provided a stem cell which maintains functional capacity upon self renewal or cell division in culture.

In another aspect of the present invention, there is provided a method of culturing self renewal stem cells to maintain or enhance functional capacity upon self renewal, said method comprising subjecting said stem cells to an effective amount of a retinoid or equivalent thereof or modulating expression and/or activity of an RAR or equivalent thereof in the stem cell.

The present invention relates to methods for self renewal of stem cells. In particular the invention relates to stem cells of increased transplant potential or enhanced self renewal, stem cell cultures derived therefrom and uses of the stem cell cultures for treatment and particularly for transplantation and gene therapy protocols.

### INTRODUCTION

Hematopoiesis is an ongoing process throughout life in which blood cells are produced. There is a large number of blood cells produced daily in homeostasis, with approximately 3.0×10⁹ erythrocytes (red blood cells), 2.5×10⁹ platelets and 1.5×10⁹ granulocytes and other white blood cells as required, produced per kg body weight per day, based on a 70 kg male adult. All hematopoietic cells arise from cells termed hematopoietic stem cells, which undergo processes involving huge amplification accompanied by differentiation into the different blood cell types. These processes are thought to be regulated by the bone marrow microenvironment in which the hematopoietic cells reside, the hematopoietic cells themselves, and by growth factors and other substances that circulate throughout the microenvironment.

In recent years, an increasing number of investigators have been interested in ex vivo culture of hematopoietic precursor cells for purposes such as stem cell expansion and retroviral-mediated gene transduction. Invariably, the culture of these cells leads to a rapid decline in stem cell activity, resulting in markedly impaired transplantability of the cultured cell populations. There is a need to improve on such methods, and for gene therapy purposes using vectors such as oncoretroviral vectors, it is essential for a stem cell to divide but be prevented from differentiating during the culture period in order to maintain its stem cell potential and accordingly, enhance the possibility of correcting a genetic deficiency in hematopoietic stem cells.

An object of the present invention is to overcome or at least alleviate some problems of the prior art and to provide a stem cell population which at least maintains some transplant potential in cultured stem cells.

### SUMMARY OF THE INVENTION

In a first aspect of the present invention there is provided a stem cell which maintains functional capacity upon self renewal or cell division.

In another aspect of the present invention, there is provided a method of culturing self renewal stem cells to maintain or enhance functional capacity upon self renewal, said method comprising subjecting said stem cells to an effective amount of a retinoid or equivalent thereof.

In another aspect of the present invention, there is provided a method of inducing self renewal of stem cells, said method comprising subjecting said stem cells to an effective amount of a retinoid or equivalent thereof.

In yet another aspect of the present invention there is provided a method of altering self renewal of stem cells said method comprising modulating expression and/or activity of an RAR or equivalent thereof in said stem cells.

In another aspect of the present invention there is provided a method of inducing differentiation of a stem cell, said method comprising decreasing expression and/or activity of RAR or equivalent thereof in said stem cell.

In yet another aspect of the present invention there is provided a self renewal stem cell population with enhanced functional capacity.

In another aspect of the present invention there is provided a method of transplantation of stem cells into a host animal, said method comprising:

obtaining a stem cell population;

culturing the stem cell population in the presence of an effective amount of a retinoid for a period sufficient to result in self renewal and enhanced functional capacity; and

transplanting the self renewal cells into the host animal.

In yet another aspect of the present invention there is provided a method of transplantation of stem cells into a host animal, said method comprising:

obtaining a stem cell population;

increasing expression and/or activity of a RAR or equivalent thereof in the stem cell population;

culturing the stem cell population, preferably in the presence of an effective amount of a retinoid for a period sufficient to result in self renewal and enhanced functional capacity; and

transplanting the self renewal cells into the host animal.

In another aspect of the present invention there is provided a method of gene therapy in a host animal, said method comprising:

obtaining a stem cell population;

culturing the stem cell population in the presence of an effective amount of a retinoid for a period sufficient to result in self renewal;

introducing a gene of interest into the stem cells; and

transplanting the cells into the host animal.

In another aspect of the present invention there is provided a method of tissue regeneration, said method comprising

culturing stem cells to maintain or enhance functional capacity upon self renewal, said method comprising subjecting said stem cells to an effective amount of a retinoid or equivalent thereof; and

transplanting said stem cells into a host to regenerate the tissue.

In another aspect of the present invention there is provided a method of tissue regeneration, said method comprising

altering self renewal of stem cells by modulating expression and/or activity of an RAR or equivalent thereof in said stem cells; and

transplanting said stem cells into a host to regenerate the tissue.

In another aspect the present invention provides compositions for use in the methods described herein comprising a retinoid or equivalent and a biologically acceptable carrier.

### FIGURES

FIG. 1 shows the effect of ATRA on serially transplantable hematopoietic stem cells. 2000 FACS-enriched hematopoietic precursors (lin− c-kit+ Sca-1+) were added to wells containing media and cytokines (SCF, IL-6, IL-11 and Flt-3 ligand), and cultured without (No ATRA) or with (ATRA) 1 umol ATRA. Irradiated primary Ly5.1 recipients (6 mice per group) were transplanted with 100 000 normal Ly5.1 bone marrow cells together with 1000 noncultured (Start) Ly5.2 lin− c-kit+ Sca-1+ cells or with all cells that grew from 1000 of these precursors after 3 (3D) or 7 (7D) days in liquid suspension culture. Subsequent transplants (secondary, tertiary and quaternary) were performed using fractions of bone marrow harvested from the primary, secondary or tertiary, respectively, transplant recipients. Data are expressed as the mean +/− SEM donor cell reconstitution in the peripheral blood of transplanted recipients analyzed at 6 months (primary recipients) or 3 months (secondary, tertiary and quaternary recipients) post-transplant.

FIG. 2 shows the expression of the following retinoic acid receptors in populations of hematopoietic cells, specifically lin− c-kit+ Sca-1+ cells (which contain stem cells) and lin− c-kit+ Sca-1− (which do not contain stem cells). The cells were isolated by FACS, mRNA prepared and the expression of the following retinoic acid receptors were analysed by RT-PCR methods:

A) Expression of RARα 1 and 2

B) Expression of RARβ 1, 2 and 3, and

C) Expression of RARγ 1 and 2.

FIG. 3 shows the expression of RARβ2 in populations of lin− c-kit+ Sca-1+ cells and lin− c-kit+ Sca-1− cells cultured with or without ATRA. FACS-enriched hematopoietic precursors (lin− c-kit+ Sca-1+ or lin− c-kit+ Sca-1−) were added to wells containing media and cytokines (SCF, IL-6, IL-11 and Flt-3 ligand), and cultured without (No ATRA) or with (+ATRA) 1 μmol ATRA for 3, 7 or 14 days. After culture, the cells were retrieved, mRNA prepared and expression of RAR beta 2 was analysed by RT-PCR methods. LKS+=lin− c-kit+ Sca-1+ cells, LKS−=lin− c-kit+ Sca-1− cells; D=day of culture.

FIG. 4 shows the expression of RARγ1 in populations of lin− c-kit+ Sca-1+ cells and lin− c-kit+ Sca-1− cells cultured with or without ATRA. FACS-enriched hematopoietic precursors (lin− c-kit+ Sca-1+ or lin− c-kit+ Sca-1−) were added to wells containing media and cytokines (SCF, IL-6, IL-11 and Flt-3 ligand), and cultured without (No ATRA) or with (+ATRA) 1 μmol ATRA for 3, 7 or 14 days. After culture, the cells were retrieved, mRNA prepared and expression of RAR γ 1 was analysed by RT-PCR methods. LKS+=lin− c-kit+ Sca-1+ cells, LKS−=lin− c-kit+ Sca-1− cells; D=day of culture.

FIG. 5 shows the effect of overexpressing RARα or RARγ in hematopoietic cells. Bone marrow was harvested from mice at day 4 post-5-fluorouracil treatment and transduced with retroviral vectors containing no cDNA (control), RARα or RARγ cDNA. Transduced hematopoietic cells were identified by their expression of GFP and phenotyped 4 weeks after transduction. Shown are the percentage of transduced cells for each vector expressing the given phenotypical markers which identify the following cell types:

Gr-1: granulocytes; F4/80: macrophages; B220: B lymphocytes;

Thy1.2: T lymphocytes and low expression on stem cells; Ter 119: erythrocytes; and

Sca-1: associated with stem cells and activated T lymphocytes

### DESCRIPTION OF THE INVENTION

In a first aspect of the present invention there is provided a stem cell which maintains functional capacity upon self renewal or cell division.

The process of self renewal is the capacity for a stem cell to maintain its functional capacity after dividing. A loss of stem cell renewal ultimately leads to the differentiation of the stem cell into cells that have limited or no transplant potential. By “functional capacity” this is meant to refer to the ability of the stem cell to transplant and regenerate organs in a transplanted recipient. Preferably, the stem cells of the present invention have enhanced transplant potential or functional capacity. This may be demonstrated by serial transplantation capacity. The stem cells of the present invention have the ability to resist differentiation into cells of limited or no transplant potential. Continued sub-culture of stem cells often leads to the cells undergoing differentiation. It is desirable and shown in the present invention that this process can be delayed in the stem cells provided.

To demonstrate self-renewal of the stem cell, one has to be able to compare the functional capacity of the stem cell population prior to and after manipulation. This will provide an indication whether functional capacity is “maintained” or “enhanced”. To prove that the stem cells maintain a high degree of primitiveness they may be subjected to successive rounds of transplantation in serial transplant studies. If no self-renewal occurs during the manipulation of the stem cells, the manipulated stem cells will not be capable of sustaining hematopoiesis in transplanted recipients for as long as the unmanipulated stem cells. If maintenance occurs, both the unmanipulated and manipulated stem cell populations should have similar transplant potential. If self-renewal is induced during the manipulation of the stem cells, these manipulated cells should have an increased transplant potential compared to the unmanipulated stem cells. In these cases, the functional capacity is enhanced over an unmanipulated stem cell.

These studies may be proven using in vivo assays, preferably using small animal models, more preferably using the congenic mouse hematopoietic stem cell transplantation model. This model relies on the use of donor and recipient mice that can be distinguished from each other by the presence of different epitopes for the leukocyte common antigen, which is present on all white blood cells. Hence, using antibodies directed against these different epitopes, one can discriminate between hematopoietic cells generated from the “donor” population (ie the test source of cells, in this instance CD45.2+ cells) and the “host” population (ie the recipient mouse cells, in this instance CD45.1+ cells).

The stem cells of the present invention which undergo self renewal are capable of multiple transplantation and preferably do not rapidly differentiate in culture. They maintain the ability to transplant several times and may withstand transplantation of at least 3 serial transplants, preferably at least 4 serial transplants.

In a preferred aspect there is provided a stem cell which maintains functional capacity upon induction of self renewal.

Even prior to the process of self renewal, events leading to self renewal may affect the character of the cell to induce maintenance of functional capacity or differentiation. The cells of the present invention are preferably biased toward maintenance of functional capacity and hence have less of a propensity to differentiate.

In another aspect of the present invention, there is provided a method of culturing self renewal stem cells to maintain or enhance functional capacity upon self renewal, said method comprising subjecting said stem cells to an effective amount of a retinoid or equivalent thereof.

The stem cells used in the present invention may be any type of stem cell selected from the group including hematopoietic, pluripotent, somatic or embryonic stem cells. Preferably the stem cells are hematopoietic stem cells (HSC).

The sample of HSC may originate from any source including an embryonic or adult source. Preferably, the HSC source is from the bone marrow including iliac crests, tibiae, femurs, spine, periosteum, endosteum or other bone cavities. The HSC may also be derived from blood, embryonic yolk sac, fetal liver, spleen, peripheral, blood, skin, dermis, liver, or brain or may be derived from ES cells or ES cell cultures.

For isolation of bone marrow, an appropriate solution can be used to flush the bone, including, but not limited to, salt solution, conveniently supplemented with fetal calf serum (FCS) or other naturally occurring factors, in conjunction with an acceptable buffer at low concentration, generally from about 5-25 mM. Convenient buffers include, but are not limited to, HEPES, phosphate buffers and lactate buffers. Otherwise bone marrow can be aspirated from the bone in accordance with conventional techniques.

Alternatively, the stem cells may be a culture of stem cells pre-prepared by other culturing methods resulting in an undifferentiated cell culture. Methods available for such culture are known to the skilled addressee. The cells may already have been in culture for at least 3 to 7 days prior to introduction of the retinoids or equivalent thereof.

In a preferred aspect of the present invention, the method enhances functional capacity of the stem cell upon self renewal to provide a stem cell with enhanced transplant potential.

In either case, the cells can be maintained in culture or sub-cultured for self renewal to keep them in a state which favours self renewal and primitiveness and to have an ability to transplant several times.

In another aspect of the present invention, there is provided a method of inducing self renewal of stem cells, said method comprising subjecting said stem cells to an effective amount of a retinoid or equivalent thereof.

These methods described herein not only maintain the cells in a state which favours transplantation, but the cells are also cultured to induce them to renew such that they result in having maintained or enhanced functional capacity.

Throughout the description and claims of this specification, the word “comprise” and variations of the word, such as “comprising” and “comprises”, is not intended to exclude other additives, components, integers or steps.

The stem cells are subjected to a retinoid or equivalent thereof. Preferably they are subjected to the retinoids whilst in culture. The cultures of the stem cells may be freshly cultured or passaged cells having been maintained in an undifferentiated state as described above.

An “effective amount” as used herein is an amount sufficient to effect the desired result. For this aspect, the amount is that amount effective to result in self renewal and maintained or enhanced functional capacity for the stem cells. Preferably, the concentration of the retinoid or equivalent thereof is in the order of 0.1-10 μM range, preferably the concentration of retinoid or equivalent is 1 μM.

Preferably, the cells are subjected to a retinoid or equivalent thereof for a period sufficient to result in or induce self renewal and maintained or enhanced functional capacity for the stem cells. This period may be determined by measuring the self renewal capability of a particular stem cell sample as described above using a stem cell transplantation model.

Preferably, the period sufficient to subject the cells is at least 3 days. This period may be as long as 14 days. However, human cells may require a longer culture period. The period may also vary depending on the concentration of the retinoid or the equivalent thereof and the population of stem cells.

The present invention includes within its scope, the use of Vitamin A and its derivatives, all trans-retinoic acid (ATRA) and other synthetic retinoids currently available. These compounds will be collectively known as retinoids and the equivalents thereof. An equivalent is a molecule which behaves in a similar manner to the retinoid and achieves the same result as a retinoid.

Retinoids are morphogens that have major, pleiotropic effects in the regulation of embryogenesis. The diverse biologic activities of retinoids are mediated by triggering the activation of retinoic acid receptor (RAR)-retinoid X receptor (RXR) heterodimers that serve as transcription factors to regulate the expression of specific target genes. Each of the RARs and RXRs have three subtypes: α, β and γ, each of which have at least three different isoforms. In hematopoiesis, retinoids have predominantly been described as a differentiating agent, being a potent inducer of the terminal differentiation of malignant promyelocytes via activation of RARα. Applicants now show that ATRA promotes the self-renewal of highly primitive, serially transplantable cultured stem cells.

The retinoid(s) and the equivalents thereof used in the present application preferably activates an RAR. More preferably, the retinoid activates an RARγ or RARβ, more preferably RARγ, most preferably RARγ1. Most preferably the retinoid is all trans retinoic acid (ATRA) or an equivalent thereof. Preferably, ATRA stimulates all three isoforms. More preferably, ATRA activates RARγ and this may be shown by the use of RARγ specific agonists. Preferably, ATRA is exposed to the stem cells in the order of 0.1-10 μm, preferably at approximately 1 μm.

Preferably the retinoic acid is selected from the group including ATRA, 9-cis retinoic acid, or the retinoid may also be a synthetic RAR isoform-specific agonist or antagonist or any combination thereof. More preferably, the retinoid is ATRA. Combinations of different retinoids—such as ATRA+ an RAR alpha specific antagonist would predominantly stimulate RARβ and RARγ but block the activation of RAR alpha. Other combinations are within the scope of the present invention providing the combination induces the retinoid effect on RAR, or the RAR-RXR heterodimers.

In yet another aspect of the present invention there is provided a method of altering self renewal of stem cells said method comprising modulating expression and/or activity of an RAR or equivalent thereof in said stem cells.

The biological activities of retinoids (including ATRA) are mediated by triggering the activation of retinoic acid receptor (RAR)-retinoid X receptor (RXR) heterodimers that serve as transcription factors to regulate the expression of specific target genes. These receptors are encoded by a number of related genes, each of which generates distinct subtypes (designated α, β and γ). In addition, alternative splicing of each of these receptor subtypes has resulted in the generation of at least 3 isoforms of each of these subtypes. These receptors are highly conserved between species and show complex stage and tissue specific patterns of expression, thus suggesting a molecular basis for the diverse biological effects of retinoids. When considering the many possible combinations of the RAR-RXR heterodimers, with a minimum of 3 isoforms of each RAR and RXR subtype, there are more than 81 receptors through which retinoids exert their effects on cells, and activation of different receptor combinations likely mediate different biologic activities.

In a preferred aspect of the present invention there is provided a method of altering self renewal of stem cells said method comprising modulating expression and/or activity of RARγ or equivalent thereof in said stem cells. In a further preferred aspect, the RARγ is RARγ1.

Applicants have found that activation of RARγ, preferably RARγ1 is enhanced in the presence of retinoids, particularly ATRA during the process of self renewal. This activation has been found to lead to self renewal which favours maintaining or enhancing functional capacity.

In a preferred aspect of the present invention there is provided a method of altering self renewal of stem cells said method comprising modulating expression and/or activity of RARβ or equivalent thereof in said stem cells.

In yet another preferred aspect of the present invention there is provided a method of enhancing self renewal of stem cells said method comprising modulating expression and/or activity of an RAR or equivalent thereof in said stem cells.

“Altering self renewal of stem cells” includes enhancing or inhibiting or reducing self renewal and includes enhancing or inhibiting or reducing induction of self renewal. “Enhancing self renewal” is generally favourable for maintaining the cells in an undifferentiated state and hence maintains or enhances functional capacity. This is particularly useful for transplantation and gene therapy. Enhanced self renewal may result from increased activity of retinoids or RAR or increased sensitivity of the cells to retinoids by increased RAR expression or activity.

In a preferred aspect of the present invention there is provided a method of inhibiting self renewal of stem cells said method comprising modulating expression and/or activity of an RAR or equivalent thereof in said stem cells.

“Inhibiting or reducing self renewal of stem cells” favours differentiation. The cells differentiate and lose functional capacity after dividing. The cells have reduced transplant potential. Inhibited self renewal may result from decreased activity of retinoids or RAR or decreased sensitivity of the cells to retinoids by decreased RAR expression or activity.

“Modulating expression and/or activation” of an RAR as used herein includes modifying or altering the expression and/or activity of a RAR compared to an unmodified condition. That is, the invention includes an active change to the expression of a gene and/or activity of a protein that encodes the RAR to change the effect of the RAR.

Modulation of RAR expression and/or activity of RAR in the stem cell may be achieved using antagonists, inhibitors, mimetics or derivatives of the RAR protein. The terms “antagonist” or “inhibitor”, as used herein, refer to a molecule which, when bound to RAR or equivalent thereof, blocks or modulates the biological or immunological activity of RAR. Antagonists and inhibitors may include proteins, nucleic acids, carbohydrates, antibodies or any other molecules that bind to RAR. These modulators may affect the RAR directly on the cell, or they may effect the retinoid which binds to the RAR, thereby preventing activation of an RAR and hence activation of specific target genes crutial in the self renewal process.

Suitable antagonists of RAR include RAR pan (α, β, γ) which blocks the action of retinoids, particularly ATRA or RAR-specific ligands. The antagonists bind to the receptors and hence antagonise ATRA such as by competing for the receptor. Other antagonists include, but are not restricted to: single subtype-specific antagonists, such as RARα antagonist, RARβ antagonist, RARγ antagonist. RAR antagonists directed against 2 different subtypes may also be included.

Modulation may be an increase or a decrease in expression and/or activity of an RAR gene or RAR protein activity, a change in binding characteristics, or any other change in the biological, functional or immunological properties. Expression of the RAR gene may be up regulated or down regulated to affect a response by retinoids.

The term “mimetic”, as used herein, refers to a molecule, the structure of which is developed from knowledge of the structure of retinoic acid receptor or portions thereof and, as such, is able to effect some or all of the actions of RAR-like molecules.

The term “derivative”, as used herein, refers to the chemical modification of a nucleic acid encoding RAR, or the encoded RAR. A nucleic acid derivative would encode a polypeptide which retains essential biological characteristics of the natural molecule.

Modulation of RAR expression and/or activity may be achieved by direct or indirect methods. Modulation of expression and/or activity of RAR may be achieved using direct methods known to those of skill in the art and may include, but are not limited to, knockout technology, antisense technology, triple helix technology, targeted mutation, gene therapy, regulation by agents acting on transcription. Indirect methods for modulating expression and/or activity of RAR may include targeting upstream or downstream regulators. Regulators of RAR include, but are not limited to, retinoic acid and equivalents including ATRA.

Expression and/or activity of RAR may be modulated by increasing or decreasing expression of RAR DNA. Vectors incorporating cDNA encoding RAR DNA, preferably cDNA encoding RAR may be transfected into stem cells to enable overexpression of the RAR vector. Vectors include, but are not restricted to adenoviral, lentiviral, and oncoretroviral vectors.

Methods of transfection of DNA vectors into cells include those familiar to the skilled addressee. Examples of suitable methods are disclosed by Ausubel et al. Preferably, the RAR cDNA is inserted into a suitable vector for transfection into the stem cells.

The RAR as used herein is preferably RARγ or RARβ, more preferably RARγ, most preferably RARγ1. Applicants have found that self renewal is most likely due to activation of RARγ more likely, RARγ1. ATRA likely enhances self renewal via this receptor. However, RARβ may also prove to enhance self renewal and hence enhance functional capacity.

In a preferred aspect of the present invention, there is provided a method of inducing self renewal of stem cells, said method comprising increasing expression and/or activity of an RAR or equivalent thereof in said cells.

As previously discussed, applicants have found that RARγ or RARβ, preferably RARγ, more preferably RARγ1 is enhanced in stem cells which retain self renewal potential. By increasing either or both the expression of the RAR or increasing the number of RAR, the cells have improved renewal capacity. Preferably the expression and/or activity of the RAR is increased by methods described above. Most preferably, it is increased by enhancing interaction of RAR with retinoids, preferably ATRA or an equivalent thereof.

In another aspect of the present invention there is provided a method of inducing differentiation of a stem cell, said method comprising decreasing expression and/or activity of RAR or equivalent thereof in said stem cell.

Conversely, by decreasing the expression and/or activity of RAR, the effect of the retinoids on the stem cells is reduced. Applicants have found that this favours differentiation.

In yet another aspect of the present invention there is provided a self renewal stem cell population with enhanced functional capacity. Preferably, the stem cell population is prepared by the methods described herein.

The self renewal cell population of the present invention may also include modulated expression and/or activity of RAR, preferably RARγ or RARβ, more preferably RARγ, most preferably, RARγ1, to enhance self renewal as described above.

Preferably the stem cell population is a hematopoietic stem cell population with enhanced self-renewal and functional capacity.

In another aspect of the present invention there is provided a method of transplantation of stem cells into a host animal, said method comprising:

obtaining a stem cell population;

culturing the stem cell population in the presence of an effective amount of a retinoid for a period sufficient to result in self renewal and enhanced functional capacity; and

transplanting the self renewal cells into the host animal.

As used herein, the term “culturing” means to grow or maintain the cell population in a viable state. This may be accomplished in an in vitro system using standard tissue culture methods well known to the skilled artisan. Alternatively, the culturing may occur in vivo by exposing the donor to effective amounts of retinoid for a period of time. Retinoids may be administered to an animal orally for prolonged periods with little or no toxicity. In this way, stem cells can be expanded in vivo in a donor animal and then harvested for transplantation into another animal, or transplanted back into the donor animal at a later date.

The stem cell population is as described herein and is preferably a hematopoietic stem cell population. When used in this capacity, the transplantation process is preferably to achieve replacement of blood cells. However, this process may be used for transplantation of any stem cells for the regeneration of organs providing the appropriate stem cell is cultured in the presence of the retinoid prior to transplantation.

The amount and period of exposure to the retinoid is as described above.

Preferably, the retinoid is ATRA. However, equivalents and other synthetic equivalent molecules may be used providing the same effect of a retinoid is achieved.

In yet another aspect of the present invention there is provided a method of transplantation of stem cells into a host animal, said method comprising:

obtaining a stem cell population;

increasing expression and/or activity of a RAR or equivalent thereof in the stem cell population;

culturing the stem cell population, preferably in the presence of an effective amount of a retinoid for a period sufficient to result in self renewal and enhanced functional capacity; and

transplanting the self renewal cells into the host animal.

Methods of increasing expression and/or activity of a RAR or equivalent thereof in the stem cell population are as described above. The cells may be manipulated to be more receptive and respond to retinoids, preferably ATRA.

The methods of culturing the stem cells are as described above under conditions which favour self renewal or induction of self renewal in the presence of an effective amount of a retinoid, preferably ATRA. If cultured under these conditions, the stem cells should also maintain or enhance their functional capacity.

Once the cells have been exposed to the retinoid for a sufficient period, they may be transplanted by methods usually employed by the skilled addressee. Intravenous infusion is generally the favoured route of administration.

Preferably the RAR is RARγ or RARβ. More preferably the RAR is RARγ, most preferably RARγ1.

The concentration of cells may depend on the heterogeneity of the cell population. In very heterogeneous populations 5 to 10×10⁶ cells per kg body weight may be required to include sufficient stem cells for transplantation. In some human CD34⁺ populations, there may be required approximately 5×10⁶ CD34⁺ cells per kg body weight.

The frequency of transplantation will depend on the severity of the condition to be treated by transplantation. In many cases, one transplantation is required. However, in some instances, multiple cultures and infusions are necessary.

The host animal may be any animal, but is generally an animal compatible with the stem cells. Ideally, the stem cells are derived from the host animal thereby reducing the risk of immune reactions upon transplantation. Preferably the host animal is a human.

In another aspect of the present invention there is provided a method of gene therapy in a host animal, said method comprising:

obtaining a stem cell population;

culturing the stem cell population in the presence of an effective amount of a retinoid for a period sufficient to result in self renewal;

introducing a gene of interest into the stem cells; and

transplanting the cells into the host animal.

The gene of interest may be any gene, either intact or modified. Modified genes include gene sequences and nucleic acid sequences which may have deletions, additions or substitutions in the sequence which affects the DNA and protein encoded from the gene sequence. The modification will depend on the gene target or condition to be treated.

The purpose of gene therapy may be for many different reasons including:

1) correcting a hematopoietic disorder by correcting the gene in the stem cell and then transplanting the corrected stem cells into the patient

2) hematopoietic and non-hematopoietic purposes—with the evidence of plasticity (ie blood stem cells becoming cells of other organs and stem cells of other organs also having the capacity to generate different organs and tissues) there lies the potential to correct other, non-hematopoietic disorders, such as muscular dystrophy for example.

Patients with acute promyelocytic leukemia are often treated with ATRA, go into remission, then relapse. It is possible that by selectively activating only RAR αusing an RAR αagonist that these patients can be treated more effectively and without relapse which is possibly due to the self-renewal of the stem cells which then become the leukemia later on.

Other cancers (including but not restricted to other leukemias) may be treated with RAR agonists or antagonists. Whilst not being limited by theory, it is possible that if the leukemia/cancer arises at the stem cell level (even though it may not present itself as a cancerous stem cell possibly because the cancer involves transcription factors that are not used by the stem cell at that level), differentiation of the stem cell may be enhanced by using an RAR gamma antagonist thereby doing the opposite of self-renewal, to drive those cancerous stem cells to a stage where the patients can effectively be treated for the cancer (radiotherapy, chemotherapy, other therapies commonly used). Patients may then be transplanted with normal marrow cells if required.

It is possible that the presence of different retinoic acid receptors are involved in different types of leukemias. For example it is known that RAR\acute{α} is definitely involved in acute promyelocytic leukemia. Accordingly, it is contemplated that the present invention may be used as a method of diagnosis.

In another aspect of the present invention there is provided a method of tissue regeneration, said method comprising

culturing stem cells to maintain or enhance functional capacity upon self renewal, said method comprising subjecting said stem cells to an effective amount of a retinoid or equivalent thereof; and

transplanting said stem cells into a host to regenerate the tissue.

Due to the recent discovery of stem cell plasticity (for instance, hematopoietic stem cells to neural cells, neural stem cells into hematopoietic cells, liver regeneration after transplantation with hematopoietic stem cells), the stem cells may be transplanted into environments within a host which favours the differentiation of the cells to regenerate different organs. In many instances, it has been shown that the stem cells enter these organs after normal transplantation procedures (such as intravenous infusion) due to the capacity of the stem cells to home and lodge into different tissues. However, there may be some instances where a more directed transplant will be required, such as transplantation into a specific location of the brain as in therapeutics for diseases such as Alzheimer's disease and Parkinson's disease.

In another aspect of the present invention there is provided a method of tissue regeneration, said method comprising:

altering self renewal of stem cells by modulating expression and/or activity of an RAR or equivalent thereof in said stem cells; and

transplanting said stem cells into a host to regenerate the tissue.

Altering self renewal and modulating expression and/or activity are as described above. However, this method may be used in a manner which firstly favours self renewal of the stem cells to improve transplantation potential. This may be achieved as described above by increasing expression and/or activity of RAR or equivalent thereof. Once the cells are transplanted, they may be induced to decrease the expression and/or activity of the RAR so as to induce differentiation of the cells. This may be achieved by genetically manipulating the stem cells with promoters that can be switched on or off depending the result required.

In another aspect the present invention provides compositions for use in the methods described herein comprising a retinoid or equivalent and a biologically acceptable carrier. Preferably, the retinoid is ATRA.

Examples of the procedures and aspects in the present invention will now be more fully described. It should be understood, however, that the following description is illustrative only and should not be taken in any way as a restriction on the generality of the invention described above.

### EXAMPLES

### Example 1

### Demonstration of the Self-Renewal Capacity of Hematopoietic Cells Cultured with Retinoids

Irradiated CD45.1+ primary murine recipients were transplanted with 1×10⁵ normal CD45.1+ bone marrow cells together with 1000 freshly isolated lin− c-kit+ Sca-1+ CD45.2+ (LKS) murine hematopoietic precursors or with all cells that grew from 1000 LKS after 3 or 7 days of culture with or without 1 μM ATRA. Primary recipients were euthanased 6 months post-transplant, their femoral marrows flushed and pooled within treatment groups and bone marrow from 1/10th of a femur of these mice were injected into each secondary irradiated recipient. Subsequent serial transplants were likewise performed at 4 month intervals post-transplant, with the 5th serial transplant currently being assessed. Recipient mice were analysed for multilineage repopulating donor cells (>5% CD45.2+) in each round of transplants.

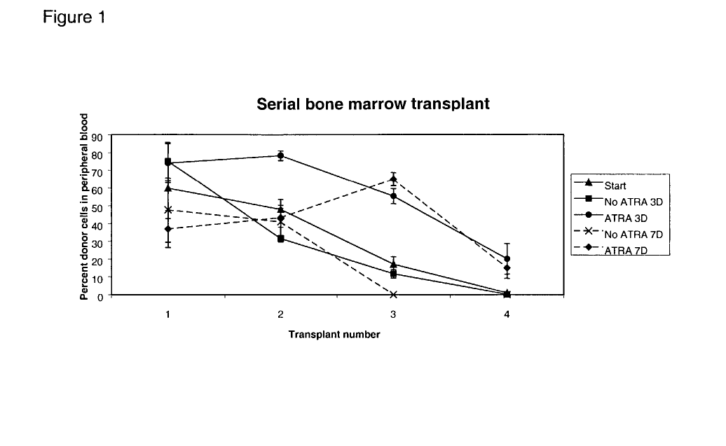

All serial transplant recipients of both the 3 day and 7 day ATRA-treated LKS had significant multilineage donor reconstituting ability, with donor repopulating levels of quaternary transplant recipients being 20.09±8.63% and 14.98±5.94% respectively (FIG. 1). In marked contrast, none of the quaternary transplant recipients of the freshly isolated (noncultured) LKS or LKS cultured without ATRA for 3 days had detectable donor cell reconstitution, and LKS cultured without ATRA for 7 days did not have any detectable repopulating activity in tertiary recipients (FIG. 1). Throughout the successive rounds of transplants there was no significant difference in marrow cellularity in any of the transplanted mice of any of the treatment groups, nor was there any evidence of leukemia in the recipient mice of the ATRA-treated LKS.

### Example 2

### Investigation of the Key Retinoic Acid Receptors Through Which ATRA is Enhancing the Self-Renewal of Hematopoietic Stem Cells

This effect of ATRA on the hematopoietic stem cells is markedly different to its differentiation-inducing effects on more mature granulocyte precursor cells. Using an RAR pan (α, β and γ) antagonist that competitively blocks the actions of ATRA on RARs, it has been previously demonstrated that the effects of ATRA on hematopoietic stem cells are mediated through the RARs (2). Hence this experiment focussed on determining which of the RARs are important in the effects of ATRA on hematopoietic stem cells.

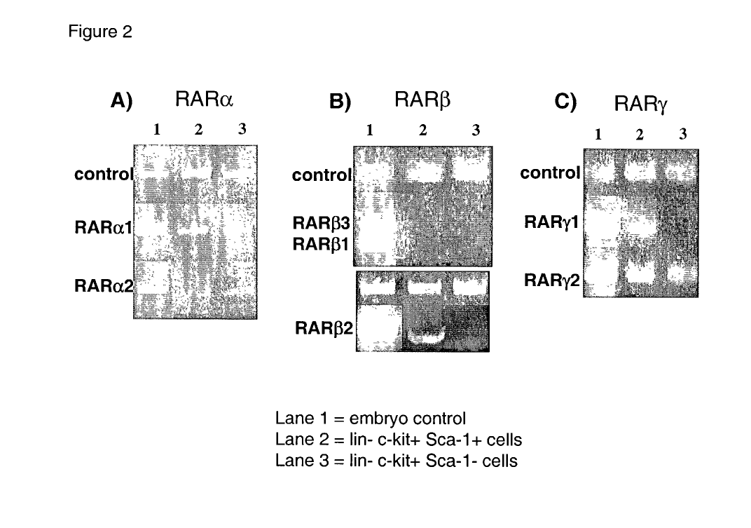

In order to determine the mechanisms whereby which ATRA is promoting stem cell self-renewal, RT-PCR methods were used to analyse the expression of different RARs by distinct populations of hematopoietic cells ranging from very primitive populations of cells containing stem cells to the most mature blood cells. In addition, by comparing the expression profiles of the hematopoietic populations lin− c-kit+ Sca-1+ cells (which contain stem cells) vs. lin− c-kit+ Sca-1− cells (which do not contain stem cells and do not respond to ATRA in the same way as the lin− c-kit+ Sca-1+ cells), the most striking differences between the populations have been the expression of RARβ2 and RARγ1 by lin− c-kit+ Sca-1+ cells but not by lin− c-kit+ Sca-1− cells (FIG. 2).

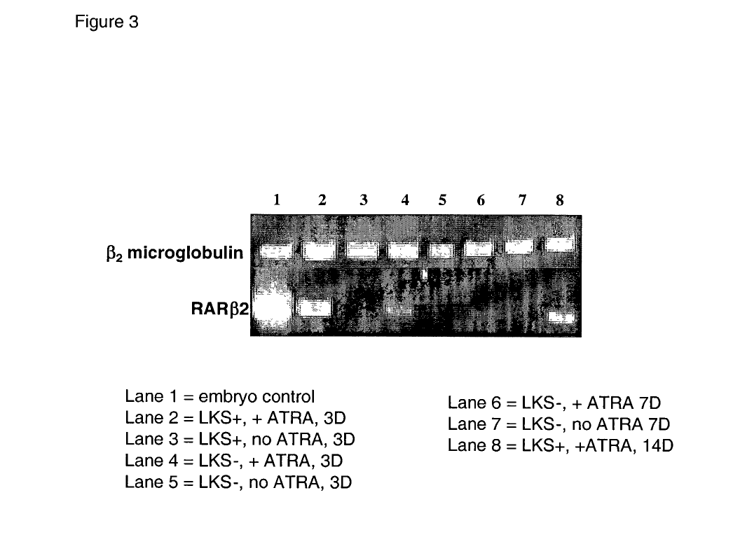

Consideration was then given to the expression of the different RARs by progeny derived from lin− c-kit+ Sca-1+ cells (LKS+) or lin− c-kit+ Sca-1− cells (LKS−) after they had been cultured with or without ATRA for 3, 7 and 14 days. We have shown that LKS+ cells cultured with ATRA for 3 and 7 days show functional self-renewal (FIG. 1), and we have previously shown that we can culture LKS+ cells for up to 14 days and still retain some transplant potential (ref 2), hence these time points were suitable to examine expression of the different RARs in cell populations that contained stem cells at these time points. The most striking differences between the populations were in their expression profiles of RARβ2 and RARγ1, both of which have been shown to be expressed by LKS+ cells but not LKS− cells (FIG. 2). RARβ2 was expressed by cells derived from ATRA-treated LKS+ and LKS− (FIG. 3), suggesting that ATRA-treatment upregulates RARβ2 in cultured cells. This effect is not likely to be associated with a stem cell specific effect, as LKS− cells do not contain stem cells and do not self-renew in response to ATRA treatment.

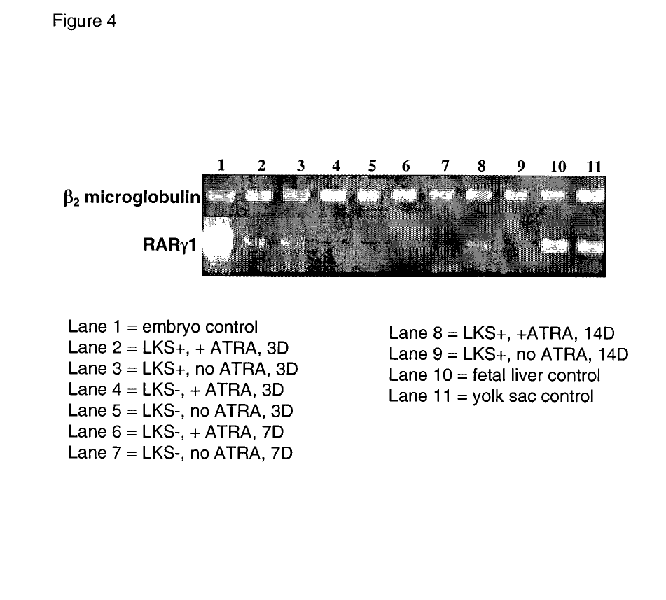

In contrast, RARγ1 expression was maintained in cultures of ATRA-treated LKS+ cells, but rapidly declined in LKS+ cells cultured without ATRA (FIG. 4). In addition, RARγ1 was not expressed in ATRA-treated LKS− cells, hence the expression of RARγ1 was considered to be more associated with the cell population being treated with ATRA, that is, the stem cells contained within the LKS+ cells.

To establish whether RARγ1 has a role in hematopoietic stem cell self-renewal, RARγ was overexpressed in primary bone marrow cells using retroviral-mediated gene transfer. The same retroviral vector was modified and utilized for these studies, and all hematopoietic cells transduced by these vectors expressed green fluorescence protein (GFP), which was detectable by flow cytometry analysis. The control retroviral vector did not contain any cDNA (hence the transduced cells did not overexpress anything). To overexpress RARα, RARα cDNA was inserted into the retroviral vector. Likewise, to overexpress RARγ, RARγ cDNA was inserted into the retroviral vector.

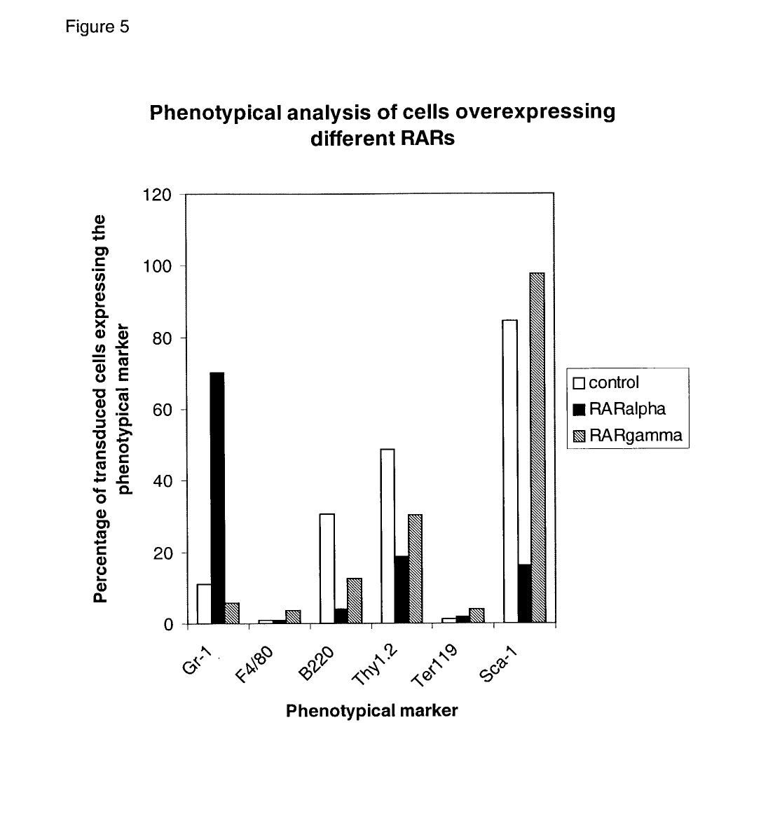

Four weeks after the hematopoietic cells were transduced, they were phenotyped utilizing antibodies directed against different hematopoietic cell type-specific antigens and analysed by flow cytometry. The RARγ overexpressing cells lacked lineage marker expression and expressed high levels of the stem cell-associated antigen, Sca-1 (FIG. 5). In contrast, bone marrow cells that overexpressed RARα were predominantly cells of the granulocytic lineage, with the majority of cells expressing Gr-1 (FIG. 5). The control cells contained a mixture of all cell types (FIG. 5).

### REFERENCES

1. Ausubel F M, Brent R. Kingston R E, Moore D D, Seidman J G, Smith J A, Struhl K (Eds). Current Protocols in Molecular Biology. Chapter 9: The Introduction of DNA into Mammalian Cells. John Wiley and Sons Inc.

2. Purton L E, Bernstein I D, Collins S J. (1999). All-trans retinoic acid delays the differentiation of primitive hematopoietic precursors (lin− c-kit+ Sca-1+) while enhancing the terminal maturation of committed granulocyte/monocyte progenitors. Blood 94:483-495.

3. Purton L E, Bernstein I D, Collins S J. (2000). All-trans retinoic acid enhances the maintenance of long-term repopulating hematopoietic stem cells. Blood 95:470-477.

Finally it is to be understood that various other modifications and/or alterations may be made without departing from the spirit of the present invention as outlined herein.

1. A stem cell which maintains functional capacity upon self renewal or cell division in culture.

2. A stem cell according to claim 1 having enhanced transplant potential or functional capacity.

3. A stem cell according to claim 1 or 2 having the ability to undergo at least 3 transplantations.

4. A stem cell according to any one of claims 1 to 3 which maintains functional capacity upon induction of self renewal.

5. A stem cell according to any one of claims 1 to 4 selected from the group including hematopoietic, pluripotent, somatic or embryonic stem cells.

6. A stem cell according to claim 5 wherein the cell is a haematopoietic stem cell.

7. A method of culturing self renewal stem cells to maintain or enhance functional capacity upon self renewal, said method comprising subjecting said stem cells to an effective amount of a retinoid or equivalent thereof.

8. A method of inducing self renewal of stem cells, said method comprising subjecting said stem cells to an effective amount of a retinoid or equivalent thereof.

9. A method according to claim 7 or 8 wherein the cell is selected from the group including hematopoietic, pluripotent, somatic or embryonic stem cells.

10. A method according to claim 9 wherein the cell is a haematopoietic stem cell.

11. A method according to any one of claims 7 to 10 wherein the cells are derived from a pre-cultured undifferentiated cell culture.

12. A method according to claim 11 wherein the cells are pre-cultured for 3 to 7 days.

13. A method according to any one of claims 7 to 12 for enhancing transplant potential.

14. A method according to any one of claims 7 to 13 wherein the retinoid or equivalent thereof is selected from the group including vitamin A, all trans-retinoic acid (ATRA), 9-cis retinoic acid, synthetic retinoids, synthetic RAR isoform-specific agonists or antagonists or any combination thereof.

15. A method according to claim 14 wherein the retinoid is ATRA.

16. A method according to any one of claims 7 to 15 wherein the retinoid or equivalent thereof activates an RAR.

17. A method according to claim 16 wherein the RAR is RARγ or RARβ.

18. A method according to claim 16 or 17 wherein the RAR is RARγ.

19. A method according to claim 18 wherein the RARγ is RARγ1.

20. A method according to any one of claims 7 to 19 wherein the stem cells are subjected to 0.1 to 10 μM retinoid or equivalent thereof.

21. A self renewal stem cell population prepared by the method according to any one of claims 7 to 20.

22. A method of altering self renewal of stem cells said method comprising modulating expression and/or activity of an RAR or equivalent thereof in said stem cells.

23. A method according to claim 22 wherein the altering of self renewal includes enhancing, inhibiting or reducing self renewal.

24. A method according to claim 22 or 23 wherein the RAR is RARγ or an equivalent thereof.

25. A method according to claim 24 wherein the RARγ is RARγ1.

26. A method according to claim 22 or 23 wherein the RAR is RARβ or equivalent thereof.

27. A method according to any one of claims 23 to 26 wherein the self renewal is inhibited by inhibiting expression and/or activity of RAR or equivalent thereof.

28. A method according to claim 27 wherein the expression and/or activity of RAR or equivalent thereof is inhibited by an antagonist of RAR selected from the group including RAR pan (α, β, γ), RAR-specific ligands, RARα antagonists, RARβ antagonists, RARγ antagonists or a combination thereof.

29. A method according to claim 27 wherein the expression and/or activity of RAR or equivalent thereof is inhibited by inhibiting an RAR gene.

30. A method according to any one of claims 23 to 26 wherein the self renewal is enhanced or induced by increasing expression and/or activity of RAR or equivalent thereof.

31. A method according to claim 30 wherein the expression and/or activity of RAR or equivalent thereof is increased by subjecting the cell to a retinoid or equivalent thereof or upregulating an RAR gene in the cell.

32. A method according to claim 31 wherein the retinoid or equivalent thereof is selected from the group including vitamin A, all trans-retinoic acid (ATRA) 9-cis retinoic acid, synthetic retinoids, synthetic RAR isoform-specific agonists or antagonists or any combination thereof.

33. A method according to claim 32 wherein the retinoid is ATRA.

34. A method of inducing differentiation of a stem cell, said method comprising decreasing expression and/or activity of RAR or equivalent thereof in said stem cell.

35. A stem cell population prepared by the method according to any one of claims 22 to 34.

36. A method of transplantation of stem cells into a host animal, said method comprising:
obtaining a stem cell population prepared by a method according to any one of claims 7 to 20;
culturing the stem cell population in the presence of an effective amount of a retinoid for a period sufficient to result in self renewal and enhanced functional capacity; and
transplanting the self renewal cells into the host animal.

37. A method of transplantation of stem cells into a host animal, said method comprising:
obtaining a stem cell population prepared by a method according to any one of claims 7 to 20;
increasing expression and/or activity of a RAR or equivalent thereof in the stem cell population;
culturing the stem cell population; and
transplanting the self renewal cells into the host animal.

38. A method according to claim 37 further comprising culturing the stem cell population in the presence of an effective amount of a retinoid for a period sufficient to result in self renewal and enhanced functional capacity.

39. A method of gene therapy in a host animal, said method comprising:
obtaining a stem cell population prepared by a method according to any one of claims 7 to 20;
culturing the stem cell population in the presence of an effective amount of a retinoid for a period sufficient to result in self renewal and enhanced functional capacity;
introducing a gene of interest into the stem cells; and
transplanting the cells into the host animal.

40. A method of tissue regeneration, said method comprising
culturing stem cells to maintain or enhance functional capacity upon self renewal, according to any one of claims 7 to 20; and
transplanting said stem cells into a host to regenerate the tissue.

41. A method of tissue regeneration, said method comprising:
altering self renewal of stem cells by modulating expression and/or activity of an RAR or equivalent thereof in said stem cells according to any one of claims 22 to 34; and
transplanting said stem cells into a host to regenerate the tissue.

42. A method according to claim 1 substantially as herein described with reference to the Examples.
